(19) United States
(12) Patent Application Publication
(10) Pub. No.: US 2026/0126456 A1
(43) Pub. Date: May 7, 2026

(54) LABELING AND DETECTION OF ASC SPECKS

(71) Applicant: UNM RAINFOREST INNOVATIONS, Albuquerque, NM (US)

(72) Inventors: Kathryn SANCHEZ, Albuquerque, NM (US); Gary ROSENBERG, Albuquerque, NM (US); Shanya JIANG, Albuquerque, NM (US); Kiran BHASKAR, Albuquerque, NM (US)

(21) Appl. No.: 19/379,433

(22) Filed: Nov. 4, 2025

Related U.S. Application Data
(60) Provisional application No. 63/716,293, filed on Nov. 5, 2024.

(51) Int. Cl. G01N 33/68

(52) U.S. Cl. CPC G01N 33/6896; G01N 33/6893; G01N 2800/2821; G01N 2800/2871

(57) ABSTRACT

A method of detecting ASC specks in a patient sample generally includes labeling a patient sample of cerebrospinal fluid with an anti-ASC antibody and detecting the anti-ASC antibody, thereby detecting the ASC specks in the patient sample. The method may be used to determine the risk of the patient having a neurodegenerative disorder or an inflammatory disorder.

### CROSS-REFERENCE TO RELATED APPLICATION

This application claims the benefit of U.S. Provisional Patent Application No. 63/716,293, filed Nov. 5, 2024, which is incorporated herein by reference in its entirety.

### GOVERNMENT FUNDING

This invention was made with government support under AG068077, NS083704, and NS100598 awarded by the National Institutes of Health. The government has certain rights in the invention.

### SUMMARY

This disclosure describes, in one aspect, a method of detecting ASC specks in a patient sample. Generally, the method includes labeling a patient sample of cerebrospinal fluid (CSF) with an anti-ASC antibody and detecting the anti-ASC antibody, thereby detecting the ASC specks in the patient sample.

In one or more embodiments, detecting the anti-ASC antibody comprises counting a number of ASC specks in the patient sample.

In another aspect, this disclosure describes a method of determining risk of having a neurodegenerative disorder in a patient. Generally, the method includes labeling a patient sample of cerebrospinal fluid with an anti-ASC antibody, detecting the anti-ASC antibody in the patient sample, thereby detecting any ASC specks, counting a number of ASC specks in the patient sample, and identifying that the patient is at risk of having a neurodegenerative disorder based on the number of ASC specks in the patient sample.

In one or more embodiments, the method further includes treating the patient for a neurodegenerative disorder.

In another aspect, this disclosure describes a method of determining risk of having an inflammatory disorder in a patient. Generally, the method includes labeling a patient sample of cerebrospinal fluid with an anti-ASC antibody, detecting the anti-ASC antibody in the patient sample, thereby detecting any ASC specks, counting a number of ASC specks in the patient sample, and identifying that the patient is at risk of having an inflammatory disorder based on the number of ASC specks in the patient sample.

In one or more embodiments, the inflammatory disorder is a neuroinflammatory disorder.

In one or more embodiments, the method further includes treating the patient for the inflammatory disorder.

In one or more embodiments of any aspect, flow cytometry may be used to detect the anti-ASC antibody. In one or more of these embodiments, the flow cytometry can include magnification.

In one or more embodiments of any aspect, the anti-ASC antibody can include a fluorescent label.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows a schematic representation of inflammasome activation in cerebrospinal fluid including the role of ASC.

FIG. 2 shows a schematic representation of an exemplary method of visualizing ASC specks consistent with the present disclosure. Human CSF is collected through a lumbar puncture and aliquoted. Presence of amyloids and pTau181 are measured in the CSF sample. An aliquot of the CSF sample is used for flow cytometry. Briefly, the CSF is centrifuged and incubated with goat serum. The samples are then incubated with primary rabbit ASC antibody (Adipogen Life Sciences, Inc., San Diego, CA) and washed with goat serum. The specks are then incubated with a fluorescent secondary antibody. The ASC specks are imaged through flow cytometry. A specific gating strategy is used to quantify the number of assembled ASC specks in CSF samples

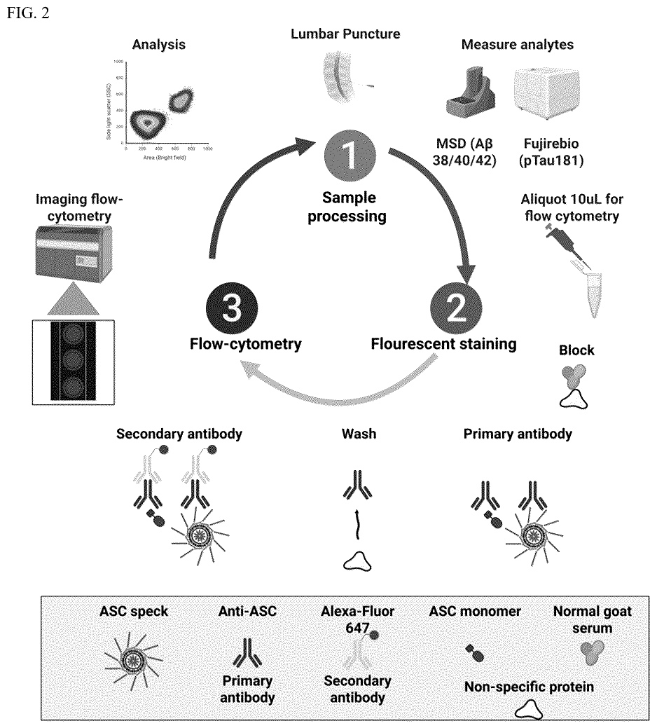

FIG. 3 shows an exemplary method of quantifying ASC-primary antibody titer. (1) ASC-cerulean macrophages were grown in petri dishes and subjected to inflammatory agents. (2) ASC was then purified from the cell lysates. More specifically, clockwise from the top of the FIG.: cells were grown to confluency in petri dishes; the inflammasome was primed and activated in the macrophages through lipopolysaccharide (LPS) and nigericin stimulation; after treatment, the cells were lysed and subjected to a series of steps including centrifugation and washes with 1×CHAPS; a 40% Percoll gradient was then used to further purify the specks; the purified specks were then stained with primary antibody and subjected to imaging flow cytometry. The number of specks detected at the 1:1000 dilution (n=3) is lower than the number detected with the 1:100 dilution (n=3). An exemplary titer of ASC specks is shown in the center. Values are reported as mean±SEM, using an unpaired Student's t-test; P=0.0544.

FIG. 4 shows representative fluorescent microscopy images of ASC specks and styrene beads. (A) Example image of ASC specks when visualized (top) and SPEEDBEADS ((Cytek Biosciences, Inc., Fremont, CA; bottom). (B) A size diagram of ASC specks and SPEEDBEADS. (C) A histogram that displays frequency of side-scatter (SSC) intensity when evaluating events that are smaller than 5 μm and in focus. Two distinct populations are observed on the histogram when sorted by SSC intensity, the ASC specks and the Speed Beads.

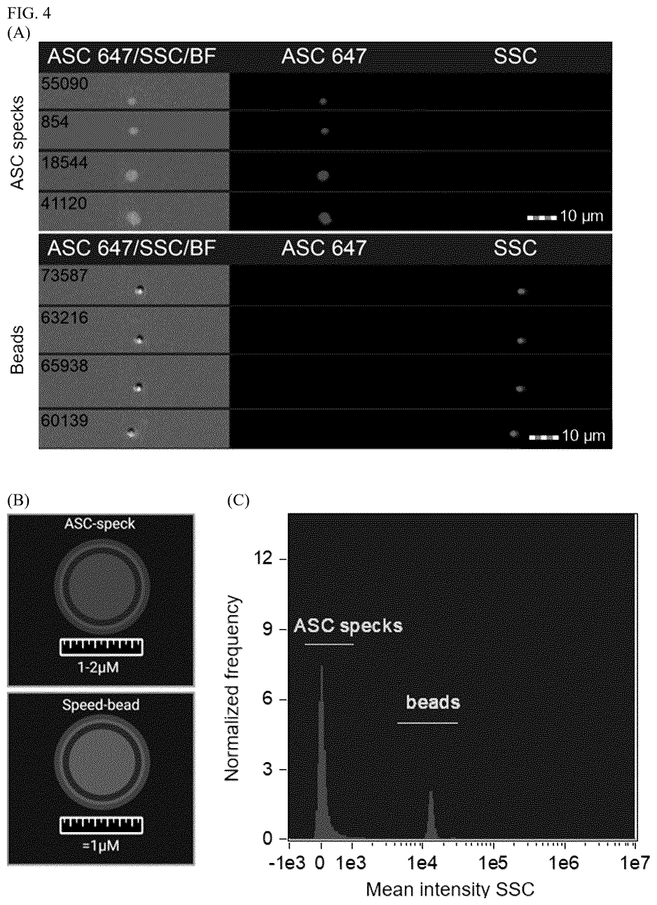

FIG. 5 shows the gating strategy used to quantify aggregated ASC specks. The graph displayed includes two distinct samples, a primary and “no primary” sample not labeled with a primary antibody. The label displays both samples; therefore, the graph displays two labels at each step. For example, events are first gated based on size. The events that are 5 μm in diameter or smaller are selected and labeled “S,S” for small specks on the histogram. This is done by constructing a scatter plot displaying the area of each brightfield channel (Area_M01_BF) on the x-axis and aspect ratio of the brightfield channel on the y-axis brightfield channel (Aspect ratio_M01_BF). These events from the “S” population are further gated by graphing a histogram that displays the gradient RMS of the brightfield channel (Gradient_RMS_M01_BF) since these events are in focus. The events that are small and in focus are further gated by generating a scatter plot that displays side scatter fluorescent intensity (SSC) and Alexa Flour 647 (ASC) intensity. This population can be identified as, “not beads,” since they have a low side scatter. Lastly, the SSC^{low} and ASC^{high} events are gated based on the ASC fluorescent intensity of the primary sample and no primary sample. Events that are above background are determined by gating the area of the histogram where the no primary sample and primary sample intersect. This effectively subtracts the area highlighted in yellow.

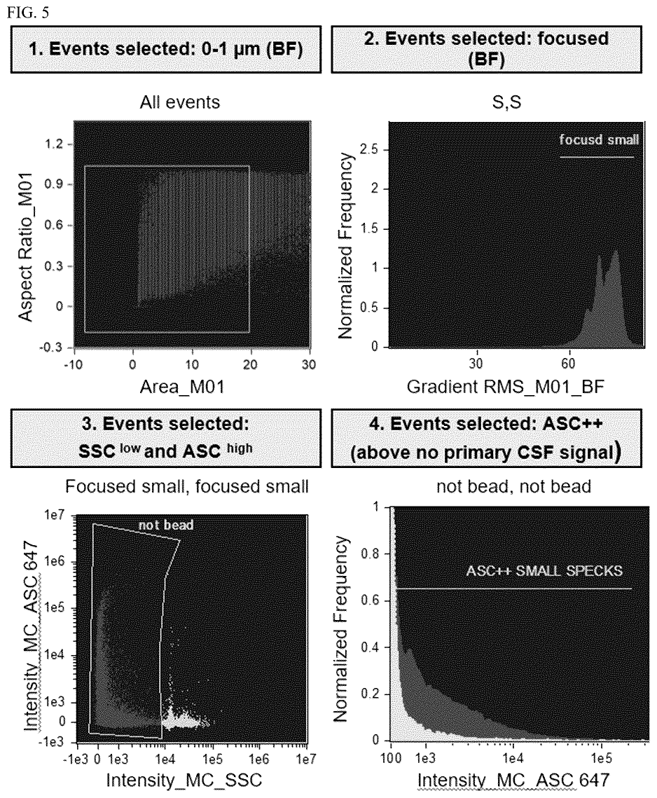

FIG. 6 shows a quantification of ASC specks detected in people not diagnosed with dementia and ASC specks detected in people known to have dementia or an associated disease. Control individuals n=11 and n=19 individuals with dementia/vascular complications. Values are reported as mean±SEM, using an unpaired Student's t-test; *, P≤0.05.

FIG. 7 shows the correlation of phosphorylated tau (p-tau) with the concentration of ASC specks detected in cerebrospinal fluid of patients with or without dementia. Pearson R=0.4177; n=9 community controls and n=28 dementia/vascular patients; *, P≤0.05.

FIG. 8 shows the correlation of amyloid-β 42/40 ratio with the concentration of ASC specks detected in cerebrospinal fluid of patients with or without dementia. Spearman R=−0.3877; n=9 community controls and n=28 dementia/vascular patients; *, P≤0.05.

FIG. 9 shows a Western blot corroborating that ASC specks are elevated in ADRD when 10 μL of sample is used. ASC-brain homogenate is shown as a negative control.

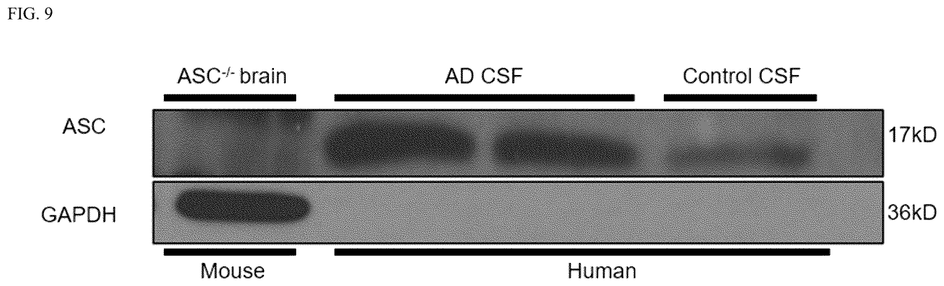

FIG. 10 shows ASC-Qβ VLPs targeting human ASC are safe and immunogenic in mice but preferentially bind to human ASC over mouse ASC. Hemoglobin, hematocrit, and blood glucose levels showing significantly altered parameters discovered during blood profiling. One-way ANOVA. *, P<0.05.

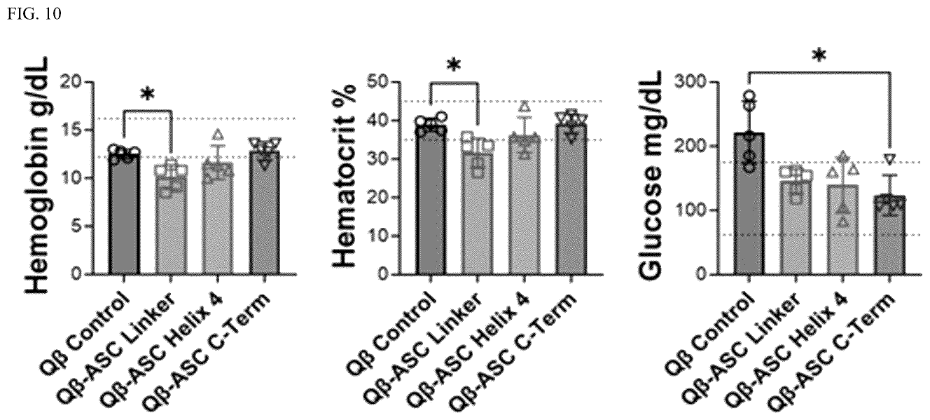

FIG. 11 shows a Western blot of human and mouse brain lysate using vaccine immune sera as the detection antibody for ASC protein demonstrating specific binding to human but not mouse ASC.

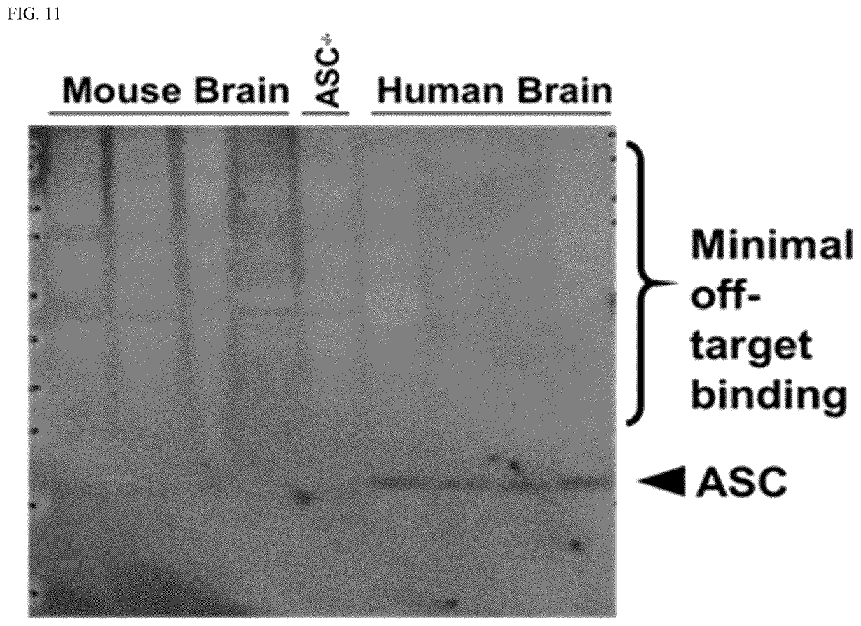

FIG. 12 shows an ROC curve showing the accuracy of CSF-ASC-speck in predicting the AD with 0.76 accuracy in the preliminary dataset. N=14 samples with mixed etiology dementia.

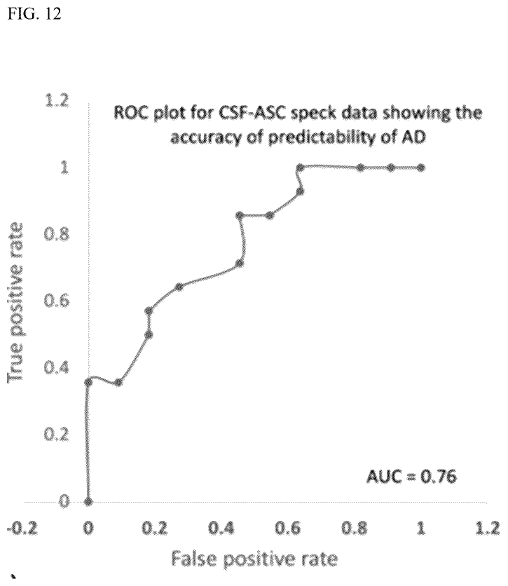

### DETAILED DESCRIPTION OF ILLUSTRATIVE EMBODIMENTS

This disclosure describes a method of detecting ASC specks in a patient sample. ASC refers to Apoptosis-associated Speck-like protein containing a caspase recruitment domain (CARD). Detecting ASC specks in a patient sample can help indicate whether the patient is at risk for developing dementia.

Dementia refers to a group of conditions characterized by impaired brain function that interfere with daily life. Dementia may be caused by numerous medical conditions or injuries, and accordingly, underlying pathologies vary between patients. Diagnosis of dementia is important for appropriate treatment. The presence of amyloid-β and tau neurofibrillary tangles in the brain are canonical pathological hallmarks of Alzheimer's disease, a common form of dementia. Additional or alternative biomarkers may improve diagnosis and treatment of patients with dementia.

The inflammasome is known to be activated in dementia. An adaptor protein component of the inflammasome, apoptosis-associated speck-like protein containing a CARD (ASC) is known to be elevated in serum and cerebrospinal fluid samples from patients with dementia.

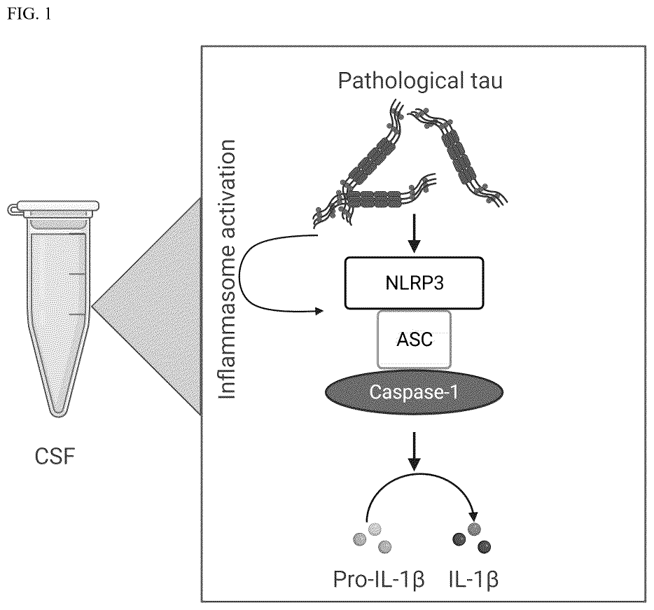

The NLRP3 inflammasome is a multiprotein complex including the protein NLRP3, which includes NACHT, LRR, and PYD domain-containing protein 3. The NLRP3 inflammasome additionally includes ASC and inflammatory caspase 1. FIG. 1 provides an exemplary schematic of the NLRP3 inflammasome. The NLRP3 inflammasome may contribute to Alzheimer's disease by promoting neuroinflammation in response to amyloid-β plaques and/or pathological tau, which includes hyperphosphorylated and aggregated tau.

ASC specks (also referred to as assembled ASC or pyroptosomes) are supramolecular assemblies that include the protein ASC and are associated with inflammation and apoptosis. In response to amyloid-β plaques or pathological tau, ASC specks are released extracellularly into the cerebrospinal fluid. ASC specks may then be taken up by immune cells and trigger a phagocytic phenotype, ultimately resulting in the release of IL-1β.

While ASC specks are present in cerebrospinal fluid of at least some patients with dementia, it is not well established whether ASC specks are present across multiple etiologies of dementia. Furthermore, ASC specks are challenging to detect in CSF using methods such as enzyme-linked immunosorbent assays (ELISA) since inflammasome-associated assays are focused on measuring IL-1β. The ASC speck is a large multiprotein complex that typically includes more than just the ASC protein. Therefore, measuring the presence of ASC specks and quantifying the number of ASC specks is technically challenging using existing methods.

The present disclosure relates to a method of detecting ASC specks in a sample, such as a patient sample. The method generally includes providing a sample, such as a patient sample, labeling the sample with an anti-ASC antibody, and detecting the anti-ASC antibody. ASC specks in the sample are therefore indirectly detected by detecting the anti-ASC antibody.

The patient can be a human or a non-human animal such as, for example, a livestock animal, a laboratory animal, or a companion animal. Examples of non-human animal patients include, but are not limited to, animals that are hominid (including, for example chimpanzees, gorillas, or orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou, or reindeer), members of the family Bison (including, for instance, bison), feline (including, for example, domesticated cats, tigers, lions, etc.), canine (including, for example, domesticated dogs, wolves, etc.), avian (including, for example, turkeys, chickens, ducks, geese, etc.), a rodent (including, for example, mice, rats, etc.), a member of the family Leporidae (including, for example, rabbits or hares), members of the family Mustelidae (including, for example ferrets), or member of the order Chiroptera (including, for example, bats).

In one or more embodiments, the sample includes cerebrospinal fluid. In one or more embodiments, the sample includes blood, plasma, serum, nasal fluid, urine, or saliva. The sample may be a solid or liquid biopsy from the nervous system, such as the central nervous system. As described herein, ASC specks are often released extracellularly, thus, ASC specks may be detected from a sample not including cells. Additionally or alternatively, ASC specks may be detected from a sample including cells. For example, the method may be used to detect ASC specks in a sample of brain cells. In one or more embodiments, providing a sample includes lysing cells to release ASC specks.

In embodiments wherein the sample includes cerebrospinal fluid, the cerebrospinal fluid may be collected using any standard method. For example, the cerebrospinal fluid may be collected using a lumbar puncture.

The method includes labeling ASC in the sample. In one or more embodiments, labeling includes contacting the sample with an antibody that binds ASC (also referred to as an “anti-ASC” antibody). As used herein, the term “antibody” refers generally to an immunoglobulin or a fragment thereof. Thus, as used herein, the term “antibody” encompasses not only immunoglobulins with an intact Fc region, but also antibody fragments capable of binding to a biological molecule (such as an antigen or receptor) or a portion thereof, including but not limited to Fab, Fab′, F(ab′)₂, pFc′, Fd, Fd′, Fv, dAB, a single domain antibody (sdAb), a variable fragment (Fv), a single-chain variable fragment (scFv) or a disulfide-linked Fv (sdFv), a diabody or a bivalent diabody, a linear antibody, a single-chain antibody molecule, or a multispecific antibody (e.g., a tribody) formed from antibody fragments. The antibody can be of any type (e.g., IgG, IgE, IgM, IgD, IgA and IgY), class (e.g., IgG1, IgG2, IgG3, IgG4, IgA1 and IgA2), or subclass.

Thus, in one or more embodiments, the anti-ASC antibody can include a humanized antibody derived from an animal single domain antibody. While an scFv has a heavy variable chain component and a light variable chain component joined by a flanking sequence, a single domain antibody consists of a single monomeric variable chain—i.e., a variable heavy chain or a variable light chain—that is capable of specifically engaging a target. A single domain antibody may be derived from an antibody of any suitable animal such as, for example, a camelid (e.g., a llama or camel) or a cartilaginous fish (e.g., a wobbegong or a nurse shark). A single domain antibody can provide superior physical stability, an ability to bind deep grooves, and increased production yields compared to larger antibody fragments.

In one or more embodiments, the antibody includes a detectable marker. The detectable marker may include a fluorescent label, a luminescent label, a colorimetric label, or a radioisotope. In one or more embodiments, the antibody includes more than one detectable marker.

The fluorescent label may be a small-molecule fluorophore or a fluorescent protein. Examples of fluorescent proteins include GFP (e.g., super folder GFP, eGFP), YFP, RFP, CFP, m Venus, tdTomato, mCherry, mKate2, and mPlum. Examples of small-molecule fluorophores include ALEXA FLUOR labels (e.g., ALEXA FLUOR 350, 405, 430, 488, 500, 514, 532, 546, 555, 568, 594, 610, 633, 635, 647, 660, 680, 700, 750, or 790; Molecular Probes, Inc., Eugene, OR), cyanine labels (e.g., CY5, CY5.5, CY3) BODIPY (Molecular Probes, Inc., Eugene, OR), coumarin and coumarin derivatives, OREGON GREEN 488 (Molecular Probes, Inc., Eugene, OR), fluorescein (FITC), tetramethylrhodamine (TRITC), TEXAS RED (Molecular Probes, Inc., Eugene, OR), Texas Red-X, allophycocyanin (APC), R-phycoerythrin (R-PE), and NOVAFLUOR dyes (e.g., NOVAFLUOR Blue, Red, or Yellow; Phitonex, Inc., Durham, NC).

The method further includes detecting the anti-ASC antibody. ASC specks are typically detected by detecting the anti-ASC antibody.

In one or more embodiments, detecting includes microscopy. As described herein, ASC specks can be very small, typically having a diameter of less than 10 μm, such as less than 6 μm. Thus, to visualize ASC specks, it may be useful to magnify the sample. In one or more embodiments, detecting includes magnifying the sample at least 4×, at least 10×, at least 20×, at least 40×, or at least 60×.

In one or more embodiments, detecting includes flow cytometry. Flow cytometry is often used to detect cells or particles with a diameter of approximately 1 μm to 15 μm. Flow cytometry may use magnification to detect smaller particles. Typically, a flow cytometer will measure side-scattered light (SSC) and forward-scattered light (FSC). SSC and FSC can be interpreted to determine the diameter of a particle. In one or more embodiments, flow cytometry includes detecting SSC of a particle. In one or more embodiments, flow cytometry includes detecting FSC of a particle. In one or more embodiments, the method further includes determining the diameter of an ASC speck using FSC and SSC measurements.

Flow cytometers can often detect labels, such as fluorophores. Detecting a fluorophore typically includes exciting the fluorophore with light, such as a laser, and detecting the light emitted by the fluorophore. To excite and detect specific fluorophores, combinations of filters may be used. Typically, flow cytometers can detect at most about ten fluorophores. In one or more embodiments, flow cytometry includes detecting a label, such as a fluorophore. In one or more embodiments, flow cytometry includes detecting more than one label. For example, flow cytometry may include detecting two fluorophores.

In one or more embodiments, flow cytometry includes imaging flow cytometry. Imaging flow cytometry refers to simultaneous analyzing particles using fluid flow and capturing multichannel images of the particles. One example of an imaging flow cytometer is the AMNIS IMAGESTREAM (Cytek Biosciences, Inc., Fremont, CA) flow cytometer.

In one or more embodiments, flow cytometry includes sorting, such as cell sorting or particle sorting. Sorted particles may be further processed, allowing one to obtain additional information about a sorted population. For example, ASC specks may be sorted out from a sample and subsequently prepared for nucleic acid sequencing, such as mRNA sequencing.

In one or more embodiments, detecting the anti-ASC antibody includes counting a number of ASC specks in the sample. To obtain an approximate concentration of ASC specks, the number of ASC specks in a predetermined volume of sample may be counted. For example, the number of ASC specks in a 5 μL sample may be counted and the total number may be divided by 5 to yield the concentration of ASC specks per μL of sample.

In one or more embodiments, ASC specks may be identified by size and/or any inflammasome sensor that has a PYD domain (e.g., NLRP1, NLRP3, AIM2) that can bind to a PYD domain of ASC. Caspase 1 also binds to ASC with CARD domain. These domains enable the protein to form a complex.

In one or more embodiments, the ASC speck has a diameter of 0.5 microns (μm) to 6 μm. The ASC speck may have a diameter of at least 0.5 μm, at least 1 μm, at least 2 μm, or at least 3 μm. The ASC speck may have a diameter of at most 6 μm, at most 5 μm, or at most 4 μm. The diameter of an ASC speck may be measured, for example, using microscopy such as fluorescence microscopy or electron microscopy.

Typically, more than one ASC speck is detected. The population of ASC specks may be described by the average ASC speck diameter. The population of ASC specks may have a mean diameter of at least 0.5 μm, at least 1 μm, at least 2 μm, or at least 3 μm. The population of ASC specks may have a mean diameter of at most 6 μm, at most 5 μm, or at most 4 μm. As used herein, “mean” may refer to the geometric mean or the arithmetic mean.

When ASC specks are measured using fluorescence, the signal intensity of an ASC speck may be meaningful. For example, an ASC speck with a high fluorescence intensity may indicate the presence of a high amount of ASC in the speck. Conversely, an ASC speck with a low fluorescence intensity may indicate a low amount of ASC in the speck. In one or more embodiments, the method includes quantifying the fluorescence intensity of an ASC speck.

In one or more embodiments, only ASC specks having a minimum fluorescence intensity may be analyzed (e.g., counted). In one or more embodiments, ASC specks may be categorized (e.g., binned) by fluorescence intensity. In one or more of these certain embodiments, counting the number of ASC specks in a sample may include counting the number of ASC specks having a categorized fluorescence intensity in the sample.

The present disclosure describes that the level of ASC specks in a sample correlates with levels of known biomarkers of pathological dementia including pathological tau and amyloid-β. Based on this observation, the present disclosure concludes that detecting ASC specks in a patient sample likely predicts risk that the patient has dementia, such as pathological dementia.

In one or more embodiments, the present disclosure relates to a method of determining whether a patient is at risk of having a neurodegenerative disorder. The method may include providing a sample from a patient, labeling the sample with an anti-ASC antibody, detecting the anti-ASC antibody, counting the number of ASC specks in the sample, and interpreting the number of ASC specks in the sample to determine whether the patient is at risk of having a neurodegenerative disorder.

In one or more embodiments, the present disclosure relates to a method of determining progression of a neurodegenerative disorder in a patient.

In one or more embodiments, the patient has or is at risk of having a neurodegenerative disorder. A neurodegenerative disorder may include, but is not limited to, Alzheimer's disease, Huntington's disease, multiple sclerosis, amyotrophic lateral sclerosis, Parkinson's disease, multiple system atrophy, Lewy body disease, Friedreich ataxia, spinal muscular atrophy, palsy (e.g., Bell's palsy, cerebral palsy), stroke, environmental nerve damage, and prior diseases. In one or more embodiments, a patient has more than one neurodegenerative disorder.

In one or more embodiments, the patient has or is at risk of having a disorder associated with neurodegeneration. For example, while heart attack (myocardial infarction) is not a classical neurodegenerative disorder, occurrence can be associated with acute neurodegeneration. Diseases associated with neurodegeneration include, but are not limited to, myocardial infarction, stroke, diabetes, viral infection, acute nerve damage, amputation, bacterial infection, cancer, and paralysis. Dysregulated IL-1 signaling can contribute to various disease states including autoinflammatory diseases (e.g., cryopyrin-associated periodic syndromes, familial Mediterranean fever), metabolic syndromes (e.g., type 2 diabetes), excessive acute inflammation (e.g. sepsis), chronic inflammatory diseases (e.g., rheumatoid arthritis, chronic obstructive pulmonary disease, gout, Alzheimer's disease), and malignancy (e.g., HER2-negative breast cancer).

Additionally, methods of the present disclosure may be suitable for use as screening tools. Thus, in one or more embodiments, the patient may not have or be at risk of having a neurodegenerative disorder. While neurodegenerative disorders are typically not identified prior to the onset of symptoms, early detection and diagnosis may result in better patient outcomes.

In one or more embodiments, the method includes comparing the number of ASC specks in a sample to a control number of ASC specks in a comparable sample. In one or more embodiments, the method includes comparing the concentration of ASC specks in a sample to a control concentration of ASC specks in a comparable sample. A comparable sample may be a sample from a subject known not to have a neurodegenerative disorder or a historic sample from the patient.

In one or more embodiments, identifying that the patient is at risk of having a neurodegenerative disorder includes identifying that the number of ASC specks in the patient sample exceeds a threshold number of ASC specks. In one or more embodiments, identifying that the patient is at risk of having a neurodegenerative disorder includes identifying that the concentration of ASC specks in the patient sample exceeds a threshold concentration of ASC specks.

In one or more embodiments, a threshold number of ASC specks in a sample may be established by determining an average number of ASC specks in a population of patients not having a neurodegenerative disorder. Typically, a threshold number of ASC specks in a sample is a number at or below which a sample is not associated with a risk of a neurodegenerative disorder, and above which a sample is associated with a risk of a neurodegenerative disorder. In one or more embodiments, a threshold concentration of ASC specks in a sample may be established by determining an average number of ASC specks in a population of patients not having a neurodegenerative disorder. Typically, a threshold concentration of ASC specks in a sample is a number at or below which a sample is not associated with a risk of a neurodegenerative disorder, and above which a sample is associated with a risk of a neurodegenerative disorder. As is described herein, an increased number of ASC specks in a sample and/or an increased concentration of ASC specks in a sample is typically associated with increased risk of neurodegenerative disorder.

In one or more embodiments, identifying that the patient is at risk of having a neurodegenerative disorder includes identifying at least 50,000, at least 75,000, at least 90,000, at least 100,000, or at least 125,000 ASC specks in the patient sample. In one or more embodiments, identifying that the patient is at risk of having a neurodegenerative disorder includes identifying at most 200,000, at most 100,000, at most 50,000, at most 40,000, at most 30,000, or at most 25,000 ASC specks in the patient sample.

In one or more embodiments, identifying that the patient is at risk of having a neurodegenerative disorder includes identifying that the patient sample has a concentration of at least 10,000, at least 15,000, at least 18,000, at least 20,000, or at least 25,000 ASC specks per μL. In one or more embodiments, identifying that the patient is at risk of having a neurodegenerative disorder includes identifying that the patient sample has a concentration of at most 40,000, at most 30,000, at most 25,000, at most 20,000, or at most 15,000 ASC specks per μL.

In one or more embodiments, the method further includes providing a control sample. The control sample may be a sample from a patient known to not have a neurodegenerative disorder. Typically, the control sample is the same type of sample as the patient sample. For example, when the patient sample is a sample of cerebrospinal fluid, the control sample is also typically a sample of cerebrospinal fluid.

In one or more embodiments, the method further includes providing a level of one or more additional biomarkers. Providing a level of one or more additional biomarkers may include measuring a level of a biomarker. For example, the method may further include measuring one or more of the proteins of the NLRP3 inflammasome, such as NLRP3 or inflammatory caspase 1.

In one or more embodiments, the method further includes providing a level of one or more biomarkers known to be associated with neurodegenerative disease. Biomarkers known to be associated with neurodegenerative disease include, but are not limited to, tau, phosphorylated tau (p-tau), amyloid-β, matrix-metalloproteases (e.g., MMP9, MMP10),

In one or more embodiments, the method further includes providing a level of one or more biomarkers associated with inflammation. Biomarkers associated with inflammation include, but are not limited to, C-reactive protein (CRP), serum amyloid A, cytokines (e.g., IL-2, IFN-α), alpha-1 glycoprotein, ceruloplasmin, hepcidin, and haptoglobin. One having skill in the art will appreciate that one biomarker may be associated with multiple phenotypes, such as inflammation and neurodegeneration.

In one or more embodiments, measuring a level of a biomarker in a sample includes detecting the biomarker using an antibody. Methods of detecting proteins using antibodies are often referred to as immunoassays. Suitable immunoassays include, but are not limited to, enzyme-linked immunosorbent assays, enzyme multiplied immunoassay techniques, DNA-based methods, such as immunoquantitative PCR (immunoPCR), electrochemiluminescent (ECL) assays, and radioactive reporter assays.

In one or more embodiments, the method further includes treating the patient for a neurodegenerative disorder. Treating a patient for a neurodegenerative disorder may include any field standard method of treatment, such as oral medication, surgery, physical therapy, cognitive therapy, or electromagnetic treatment, or passive or active immunotherapies such as vaccines. ASC specks can be used as an outcome measure to determine the effectiveness of such therapies.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

As used herein, “have,” “has,” “having,” “include,” “includes,” “including,” “comprise,” “comprises,” “comprising” or the like are used in their open-ended inclusive sense, and generally mean “include, but not limited to,” “includes, but not limited to,” or “including, but not limited to.” Further, wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, that which follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

As used herein, the word “exemplary” means to serve as an illustrative example and should not be construed as preferred or advantageous over other embodiments.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

In several places throughout the above description, guidance is provided through lists of examples, which examples can be used in various combinations. In each instance, the recited list serves only as a representative group and should not be interpreted as an exclusive list.

For any method disclosed herein that includes discrete steps, the steps may be performed in any feasible order. And, as appropriate, any combination of two or more steps may be performed simultaneously.

### EXAMPLES

The present invention is illustrated by the following examples. It is to be understood that the particular examples, materials, amounts, and procedures are to be interpreted broadly in accordance with the scope and spirit of the invention as set forth herein.

### Example 1

A sample of cerebrospinal fluid known to have ASC specks was provided. A sample of mouse brain homogenate obtained from ASC knockout mice and known to not have ASC specks (ASC−/−) was provided.

Samples were thawed on ice. A 10 μL aliquot of each sample was prepared and centrifuged at 13,500 RPM with a PRISM R Refrigerated Microcentrifuge (Labnet International, Inc., Edison, NJ). The supernatant from each sample was disposed and the pellet was kept.

50 μL of primary antibody (1:100 anti-rabbit ASC pAb AL177, AG-25B-0006-C100; Adipogen Life Sciences, Inc., San Diego, CA) diluted with 10% normal goat serum in phosphate buffered saline (PBS) was added to each sample. Samples were incubated for 30 minutes with gentle rotation. 250 μL of blocking solution (10% normal goat serum in PBS, MP Biomedicals, 191356) was added to each sample. Samples were incubated for 30 minutes with gentle rotation. Samples were centrifuged at 13500 RPM with a PRISM R refrigerated microcentrifuge (Labnet International, Inc., Edison, NJ).

50 μL of secondary antibody (1:500 goat anti-rabbit ALEXA FLUOR 647 (Molecular Probes, Inc., Eugene, OR), diluted with 10% normal goat serum in PBS) was added to each sample. Samples were incubated for 30 minutes with gentle rotation. 250 μL of blocking solution was added to each sample. Samples were incubated for 30 minutes with gentle rotation. Samples were centrifuged at 13,500 RPM with a PRISM R refrigerated microcentrifuge (Labnet International, Inc., Edison, NJ). Each pellet was resuspended in 50 μL PBS and kept on ice.

Samples were run on a AMNIS IMAGESTREAM flow cytometer (Cytek Biosciences, Inc., Fremont, CA). Samples were briefly vortexed before loading. The acquisition panel was set to 20× magnification. 5 μL of each sample was run and all events were collected without gating.

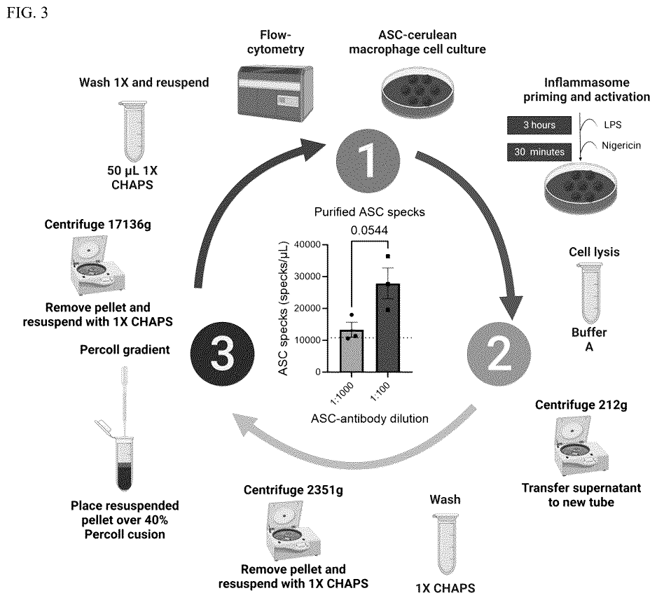

The intensity of ALEXA FLUOR 647 (Molecular Probes, Inc., Eugene, OR) was used to determine events that had a higher mean intensity (ASC++ small specks) compared to background, or a sample not stained with the primary antibody (FIG. 3). When aggregated ASC specks were quantified with flow cytometry, the mouse ASC−/− sample had a comparable number of specks to the negative (no primary antibody) control, indicated by the dashed line. The mouse ASC−/− sample also had a lower signal than 10 μL of the undiluted human CSF sample at 1:100 (n=1 sample per group, FIG. 3).

This Example established that ASC specks can be detected and quantified with AMNIS technology (Cytek Biosciences, Inc., Fremont, CA). Further, this Example established that the anti-ASC antibody was specific to ASC, and did not bind ASC−/− particles.

### Example 2

Cerebrospinal fluid samples from community members without dementia and patients with Alzheimer's disease, mixed dementia, small vessel disease, myocardial infarction, or leukoaraiosis were obtained through lumbar puncture. A summary of patient demography and biomarker levels is shown below in Table 1.

TABLE 1
Patient demography from samples used in Example 2

| | | | ASC specks | p-Tau 181 | Amyloid-β |
|---|---|---|---|---|---|
| Diagnosis | Sex | Race | (specks/μL) | (pg/mL) | 40/42 ratio |
| Control | Female | White | 33422.4 | 12 | 0.136 |
| Control | Female | White | 32988.6 | 81 | 0.116 |
| Control | Female | White | 17207.2 | 60 | 0.125 |
| Control | Female | White | 20010.2 | 25 | 0.112 |
| Control | — | White | 5677.4 | 222 | 0.122 |
| Control | Male | White | 9525.8 | 95 | 0.131 |
| Control | Female | White | 12978.4 | 55 | 0.125 |
| Control | — | White | 4567 | 37 | 0.121 |
| Control | Male | White | 13917 | 73 | 0.087 |
| Control | Female | White | 12197.4 | 100 | 0.116 |
| Control | Female | White | 5800.2 | 27 | 0.109 |
| Control | Female | White | 7672.8 | 27 | 0.063 |
| Control | Male | White | 697.8 | 71 | 0.032 |
| AD | Male | White | 11949.6 | 64 | 0.036 |
| AD | Male | White | 11968.6 | 38 | 0.091 |
| AD | Male | White | 17303.6 | 141 | 0.036 |
| AD | Male | White | 25570.2 | 65.5 | 0.024 |
| AD | Female | White | 22278 | 68 | 0.047 |
| AD | Male | White | 30886.6 | 114 | 0.047 |
| AD | Female | White | 17989.6 | 124 | 0.049 |
| AD | Female | White | 33052.2 | 136 | 0.021 |
| AD | Female | White | 15716.8 | 50 | 0.056 |
| AD | Female | White | 6851 | 150 | 0.048 |
| AD | Male | White | 20450.4 | 74.119 | 0.047 |
| MIXED | Female | White | 13245.2 | 78 | 0.076 |
| Stroke | Female | White | 20683 | 98.07 | 0.044 |
| AD | Female | White | 24546.2 | 163.069 | 0.033 |
| LA | Male | White | 28800 | 122.151 | 0.034 |
| AD | Female | White | 24183.4 | 87.151 | 0.036 |
| SVD | Male | White | 15659.2 | 76.232 | 0.036 |
| AD | Male | White | 2590.8 | 82.736 | 0.02 |

“AD” = Alzheimer's disease, “MIXED” = mixed dementia, “LA” = leukoaraiosis, “SVD” = small vessel disease.

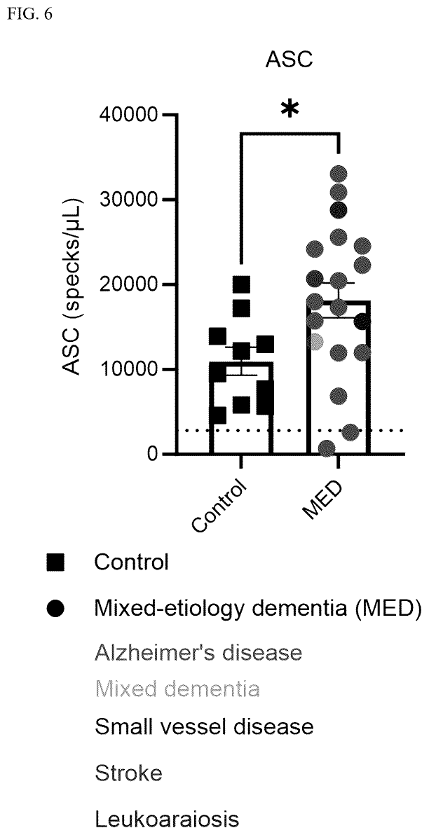

Samples were labeled with primary and secondary antibody as described in Example 1. Samples were run on an AMNIS IMAGESTREAM (Cytek Biosciences, Inc., Fremont, CA) flow cytometer as described in Example 1. For each sample, the total number of ASC specks detected in a 5 μL aliquot was counted. FIG. 6 shows a statistically significant difference in the number of ASC specs/μL detected for control samples and samples from patients with dementia.

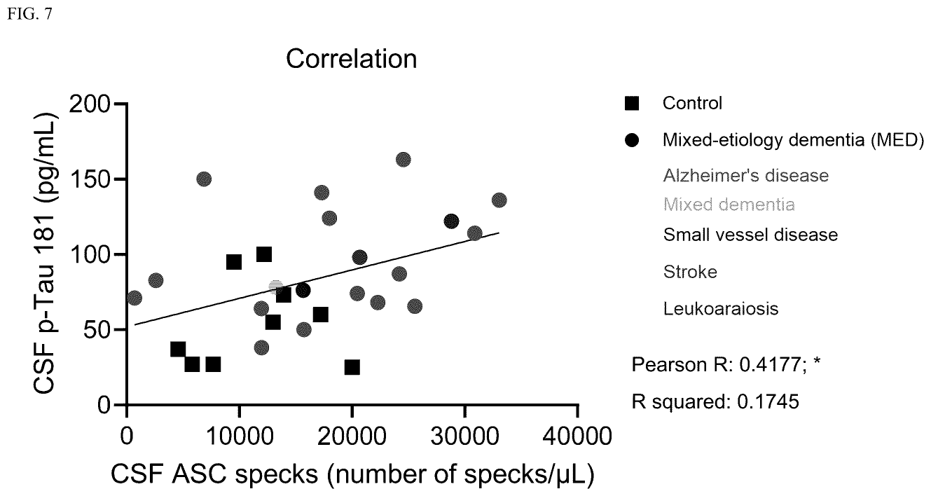

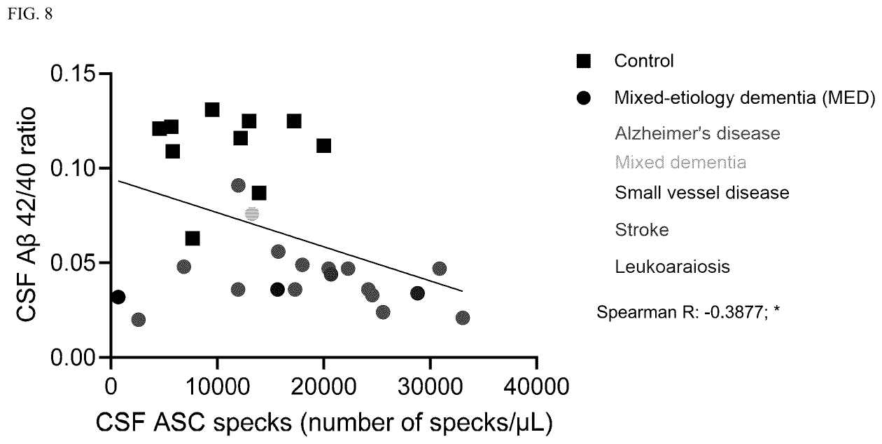

In addition, the number of ASC specks detected correlated with levels of p-Tau 181 and the amyloid-β 42/40 ratio for each sample (FIG. 7, FIG. 8). This Example established that ASC specks correlate with biomarkers of pathological dementia. In particular, ASC speck levels correlate with pathological tau levels in cerebrospinal fluid samples from patients with dementia.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method of detecting ASC specks in a patient sample, the method comprising:
providing a patient sample of cerebrospinal fluid;
labeling the patient sample with an anti-ASC antibody; and
detecting the anti-ASC antibody, thereby detecting the ASC specks in the patient sample.

2. The method of claim 1, wherein detecting the anti-ASC antibody comprises counting a number of ASC specks in the patient sample.

3. A method of determining risk of having a neurodegenerative disorder in a patient, the method comprising:
providing a patient sample from the patient;
labeling the patient sample with an anti-ASC antibody;
detecting the anti-ASC antibody in the patient sample, thereby detecting any ASC specks;
counting a number of ASC specks in the patient sample; and
identifying that the patient is at risk of having a neurodegenerative disorder based on the number of ASC specks in the patient sample.

4. A method of determining risk of having an inflammatory disorder in a patient, the method comprising:
providing a patient sample from the patient;
labeling the patient sample with an anti-ASC antibody;
detecting the anti-ASC antibody in the patient sample, thereby detecting any ASC specks;
counting a number of ASC specks in the patient sample; and
identifying that the patient is at risk of having an inflammatory disorder based on the number of ASC specks in the patient sample.

5. The method of claim 4, wherein the inflammatory disorder is a neuroinflammatory disorder.

6. The method of claim 3, further comprising:
providing a control sample from a control subject without a neurodegenerative disorder;
labeling the control sample with the anti-ASC antibody;
detecting the anti-ASC antibody in the control sample; and
counting a number of ASC specks in the control sample;
wherein identifying that the patient is at risk of having a neurodegenerative disorder comprises identifying a higher number of ASC specks in the patient sample than the control sample.

7. The method of claim 1, wherein detecting the anti-ASC antibody comprises flow cytometry.

8. The method of claim 7, wherein flow cytometry comprises magnification.

9. The method of claim 7, wherein flow cytometry comprises using an imaging flow cytometer.

10. The method of claim 1, wherein the anti-ASC antibody comprises a fluorescent label.

11. The method of claim 10, further comprising quantifying a fluorescence intensity of the ASC specks.

12. The method of claim 3, wherein identifying that the patient is at risk of having a neurodegenerative disorder comprises identifying at least 75,000 ASC specks in the patient sample.

13. The method of claim 3, wherein identifying that the patient is at risk of having a neurodegenerative disorder comprises identifying a concentration of at least 15,000 ASC specks per μL of patient sample.

14. The method of claim 3, further comprising measuring a level of one or more additional biomarkers associated with neurodegenerative disease.

15. The method of claim 14, wherein the one or more additional biomarkers associated with neurodegenerative disease comprises phosphorylated tau and/or amyloid-β.

16. The method of claim 3, further comprising treating the patient for a neurodegenerative disorder.

17. The method of claim 4, wherein identifying that the patient is at risk of having an inflammatory disorder comprises identifying at least 75,000 ASC specks in the patient sample.

18. The method of claim 4, wherein identifying that the patient is at risk of having an inflammatory disorder comprises identifying a concentration of at least 15,000 ASC specks per μL of patient sample.

19. The method of claim 4, further comprising measuring a level of one or more additional biomarkers associated with the inflammatory disorder.

20. The method of claim 4, further comprising treating the patient at risk of having the inflammatory disorder.
